(19) United States
(12) Patent Application Publication
(10) Pub. No.: US 2025/0259714 A1
(43) Pub. Date: Aug. 14, 2025

(54) DETERMINATION METHOD, QUALITY ASSURANCE METHOD, ELECTROLYSIS DEVICE, AND ELECTROLYSIS METHOD

(71) Applicant: IHI Corporation, Tokyo (JP)

(72) Inventors: Hiroaki OHARA, Tokyo (JP); Toshiyuki SUDA, Tokyo (JP); Toshiro FUJIMORI, Tokyo (JP)

(73) Assignee: IHI Corporation, Tokyo (JP)

(21) Appl. No.: 19/192,041

(22) Filed: Apr. 28, 2025

Related U.S. Application Data
(63) Continuation of application No. PCT/JP2023/038103, filed on Oct. 20, 2023.

(30) Foreign Application Priority Data
Nov. 1, 2022 (JP) 2022-175572

(51) Int. Cl. G16C 20/20; C25B 1/04; C25B 9/19; C25B 15/023; C25B 15/08

(52) U.S. Cl. CPC G16C 20/20; C25B 1/04; C25B 9/19; C25B 15/023; C25B 15/087

(57) ABSTRACT

A determination method determines whether or not target molecules including elemental hydrogen are electrolytic hydrogen-containing molecules which include: hydrogen molecules produced by water electrolysis; or molecules produced using the hydrogen molecules as a raw material. In the determination method, the method includes determining that the target molecules are the electrolytic hydrogen-containing molecules when an abundance ratio of deuterium to light hydrogen in the target molecules is less than or equal to a predetermined threshold which is smaller than an abundance ratio of deuterium to light hydrogen in nature.

### CROSS-REFERENCE TO RELATED APPLICATIONS

This application is a continuation application of International Application No. PCT/JP2023/038103, filed on Oct. 20, 2023, which claims priority to Japanese Patent Application No. 2022-175572, filed on Nov. 1, 2022, the entire contents of which are incorporated by reference herein.

### BACKGROUND

### 1. Technical Field

The disclosure relates to a determination method, a quality assurance method, an electrolysis device, and an electrolysis method.

### 2. Description of the Related Art

Carbon dioxide has been regarded as a cause of global warming, and there has been a worldwide movement to curb carbon dioxide emissions. Hydrogen is attracting attention as an alternative to fossil fuels because it does not emit carbon dioxide when used, and also because it can be obtained by electrolyzing water using renewable energy. As a method for electrolyzing water to produce hydrogen, an alkaline type water electrolysis device disclosed in WO 2019/181662 is known.

### SUMMARY

Hydrogen is conventionally produced industrially by steam-reforming fossil fuels, such as natural gas. However, there is no way to confirm that any give sample of hydrogen has been produced by water electrolysis. Ammonia is expected to be used as a next-generation fuel, and hydrocarbons are used as raw materials for various chemical products. Molecules thereof can be produced using hydrogen as a raw material, but as with hydrogen, there is no method to confirm that hydrocarbons are produced by water electrolysis. When it is possible to confirm whether these molecules are produced via water electrolysis, the quality of these molecules can be assured.

Accordingly, it is an object of the present disclosure to provide a determination method and a quality assurance method that can confirm whether target molecules are: hydrogen which has been produced by water electrolysis; or molecules which have been produced using the hydrogen as a raw material. Also, it is an object of the present disclosure to provide an electrolysis device and an electrolysis method that can easily implement these methods.

A determination method according to the present disclosure is a determination method for determining whether or not target molecules including elemental hydrogen are electrolytic hydrogen-containing molecules which include: hydrogen molecules produced by water electrolysis; or molecules produced using the hydrogen molecules as a raw material. The method includes determining that the target molecules are the electrolytic hydrogen-containing molecules when an abundance ratio of deuterium to light hydrogen in the target molecules is less than or equal to a predetermined threshold which is smaller than an abundance ratio of deuterium to light hydrogen in nature.

The target molecules may be hydrogen molecules, ammonia, or a hydrocarbon.

A quality assurance method according to the present disclosure is a quality assurance method for assuring that target molecules including elemental hydrogen are electrolytic hydrogen-containing molecules which include: hydrogen molecules produced by water electrolysis; or molecules produced using the hydrogen molecules as a raw material. The quality assurance method includes assuring that the target molecules are the electrolytic hydrogen-containing molecules when an abundance ratio of deuterium to light hydrogen in the target molecules is less than or equal to a predetermined threshold which is smaller than an abundance ratio of deuterium to light hydrogen in nature.

An electrolysis device according to the present disclosure includes: an electrolytic cell that electrolyzes water; a circulation flow path where water to be electrolyzed in the electrolytic cell circulates; a water supply flow path that supplies pure water to the circulation flow path; and a drainage flow path that drains part or all of the water in the circulation flow path, downstream of the electrolytic cell and upstream of the water supply via the water supply flow path. In the electrolysis device, an abundance ratio of deuterium to light hydrogen in hydrogen molecules produced by water electrolysis in the electrolytic cell is smaller than an abundance ratio of deuterium to light hydrogen in nature.

A flow rate control device that controls an amount of water drained in the circulation flow path may be provided in the drainage flow path.

Water supplied to the electrolytic cell may be alkaline water, and the electrolysis device may further include a membrane separator that is provided in the drainage flow path and may include a permeable membrane which selectively passes water in the alkaline water therethrough.

An electrolysis method according to the present disclosure includes: an electrolysis step of electrolyzing water in an electrolytic cell; a water supply step of supplying pure water to a circulation flow path that circulates water to be electrolyzed in the electrolytic cell; and a drainage step of draining part or all of the water in the circulation flow path, downstream of the electrolytic cell and upstream of the water supply in the water supply step. In the electrolysis method, an abundance ratio of deuterium to light hydrogen in hydrogen molecules produced by water electrolysis in the electrolytic cell is smaller than an abundance ratio of deuterium to light hydrogen in nature.

At least one selected from the group consisting of: a ratio of an amount of water consumed by electrolysis in the electrolytic cell to an amount of water supplied to the electrolytic cell; a ratio of an abundance ratio of deuterium to light hydrogen in water supplied to the electrolytic cell to an abundance ratio of deuterium to light hydrogen in hydrogen molecules produced in the electrolytic cell; and a ratio of a flow rate of water drained in the drainage step to a flow rate of water discharged from the electrolytic cell, may be controlled.

A determination device includes a determination unit that determines whether or not target molecules including elemental hydrogen are electrolytic hydrogen-containing molecules which include hydrogen molecules produced by water electrolysis, or molecules produced using the hydrogen molecules as a raw material. The determination unit determines that the target molecules are the electrolytic hydrogen-containing molecules when an abundance ratio of deuterium to light hydrogen in the target molecules is less than or equal to a predetermined threshold which is smaller than an abundance ratio of deuterium to light hydrogen in nature.

The determination device includes a measurement unit that measures the abundance ratio of deuterium to light hydrogen in the target molecules. The determination device includes a determination unit that determines that the target molecules are the electrolytic hydrogen-containing molecules when the abundance ratio of deuterium to light hydrogen in the target molecules obtained by the measurement unit is less than or equal to a predetermined threshold which is less than the abundance ratio of deuterium to light hydrogen in nature. The determination device includes an output unit that outputs a determination result determined by the determination unit.

According to the present disclosure, it is possible to provide a determination method and a quality assurance method that can confirm whether target molecules are hydrogen which has been produced by water electrolysis, or molecules produced using the hydrogen as a raw material. According to the present disclosure, it is also possible to provide an electrolysis device and an electrolysis method that can easily implement these methods.

### BRIEF DESCRIPTION OF THE DRAWINGS

FIG. 1 is schematic diagram illustrating an example of a low temperature type water electrolysis device according to the present embodiment.

FIG. 2 is a schematic diagram illustrating an example of a PEM type water electrolysis device.

FIG. 3 is a schematic diagram illustrating an example of an alkaline type water electrolysis device.

FIG. 4 is a schematic diagram illustrating an example of an AEM type water electrolysis device.

FIG. 5 is a schematic diagram illustrating an example of a medium-high temperature steam electrolysis device according to the present embodiment.

FIG. 6 is a schematic diagram illustrating an example of an SOEC type water electrolysis device.

FIG. 7 is a schematic diagram illustrating an example of a PCEC type water electrolysis device.

FIG. 8 is a schematic diagram illustrating an example of an electrolysis device including a permeation device.

### DESCRIPTION OF THE EMBODIMENTS

Some exemplary embodiments will be described with reference to the drawings. Note that dimensional ratios in the drawings are exaggerated for convenience of the description and are sometimes different from actual ratios.

A determination method according to the present embodiment determines whether or not target molecules are electrolytic hydrogen-containing molecules. The electrolytic hydrogen-containing molecules include hydrogen molecules produced by water electrolysis, and molecules produced by using the hydrogen molecules as a raw material. Water electrolysis can be performed using renewable energy. When target molecules are electrolytic hydrogen-containing molecules, and water electrolysis is performed using renewable energy, it can be determined whether the target molecules are derived from renewable energy.

Target molecules are molecules including elemental hydrogen. The target molecules may be hydrogen molecules, ammonia, or a hydrocarbon. Similarly, electrolytic hydrogen-containing molecules may be hydrogen molecules, ammonia, or a hydrocarbon. Hydrogen and ammonia can be used as carbon-free fuels. Thus, instead of fossil fuels, hydrogen and ammonia can be used as fuels derived from renewable energy. Hydrocarbons can be produced using carbon dioxide as a raw material. Thus, carbon dioxide included in plant emission gas can be recovered, and the recovered carbon dioxide can be effectively used as a raw material for chemical products.

Hydrogen molecules can be produced by water electrolysis. Water electrolysis can be performed using an electrolysis device described below. The hydrogen molecules may be hydrogen gas. Molecules such as ammonia and a hydrocarbon can be produced using hydrogen molecules produced by water electrolysis as a raw material. Ammonia can be produced using hydrogen molecules as a raw material, for example, with the Haber-Bosch method. Hydrocarbons may include at least one of methane or olefin. Methane can be produced by a methanation reaction using hydrogen molecules as a raw material. Olefin (alkene) can be produced by a Fisher-Tropsch reaction using hydrogen molecules as a raw material.

It is known that there are three isotopes of elemental hydrogen present in nature: light hydrogen (¹H or H), deuterium (²H or D), and tritium (tritium: ³H or T). Light hydrogen is the most abundant isotope of elemental hydrogen in nature. Deuterium is a stable isotope of elemental hydrogen. Tritium is a radioactive isotope, and the amount of tritium present in nature is very small.

In the determination method according to the present embodiment, target molecules are determined to be electrolytic hydrogen-containing molecules when the abundance ratio of deuterium to light hydrogen in the target molecules is less than or equal to a predetermined threshold which is smaller than the abundance ratio of deuterium to light hydrogen in nature. Electrolytic hydrogen-containing molecules produced using a method described below have a smaller abundance ratio of deuterium. Therefore, when the abundance ratio of deuterium in target molecules is smaller than that in nature, the target molecules can be determined to be electrolytic hydrogen-containing molecules.

Specifically, in the determination method according to the present embodiment, deuterium, which is a stable isotope of elemental hydrogen, is used as a tracer to determine whether or not target molecules are electrolytic hydrogen-containing molecules. Although details will be described below, deuterium molecules, such as HD and D₂, have a slower reaction rate than light hydrogen molecules, such as H₂. Thus, the abundance ratio of deuterium to light hydrogen in hydrogen molecules obtained by water electrolysis using this reaction rate difference is smaller than the abundance ratio of HDO and D₂O to H₂O in electrolytic cell supply water. Therefore, it is possible to determine that target molecules are electrolytic hydrogen-containing molecules when the abundance ratio of deuterium to light hydrogen in the target molecules is less than or equal to a predetermined threshold which is smaller than the abundance ratio of deuterium to light hydrogen in nature.

The abundance ratio of deuterium to light hydrogen in hydrogen molecules obtained by water electrolysis as described above is smaller than that in nature. Thus, hydrogen-containing molecules, such as ammonia or a hydrocarbon, made using hydrogen molecules produced by water electrolysis as a raw material have a similar abundance ratio of deuterium as the hydrogen molecules. In contrast, the abundance ratio of deuterium to light hydrogen in hydrogen obtained by steam-reforming fossil fuels, such as natural gas, is similar to the abundance ratio of deuterium to light hydrogen in nature. Therefore, the abundance ratio of deuterium in hydrogen-containing molecules, such as ammonia or a hydrocarbon produced by using hydrogen molecules as a raw material having a smaller deuterium ratio than that in nature, is also smaller than the ratio of deuterium in nature.

The abundance ratio of deuterium to light hydrogen in target molecules can be obtained by calculating a molar ratio of deuterium to light hydrogen included in the target molecules. Specifically, the abundance ratio of deuterium to light hydrogen in target molecules is a molar ratio of molecules including at least one deuterium atom to molecules including only light hydrogen atoms among molecules included in the target molecules. The abundance ratio of deuterium can be obtained using a mass spectrometer. The abundance ratio of deuterium can also be obtained using a mass spectrometer combined with a separation device, such as a gas chromatograph. The abundance ratio of deuterium can also be obtained using a gas chromatograph combined with a detector, such as a TCD (thermal conductivity detector).

The abundance ratio of deuterium to light hydrogen in nature is said to be 184 ppm or less. Thus, the abundance ratio of deuterium to light hydrogen in nature may be, for example, 184 ppm or less. The abundance ratio of deuterium to light hydrogen in nature may be the abundance ratio of deuterium to light hydrogen in Vienna Standard Mean Ocean Water (VSMOW). The abundance ratio of deuterium to light hydrogen in Vienna Standard Mean Ocean Water is about 155 ppm.

The above threshold may be smaller than the abundance ratio of deuterium to light hydrogen in nature. The threshold may be, for example, 120 ppm, 100 ppm, 80 ppm, 60 ppm, 40 ppm, 20 ppm, or 10 ppm. When the abundance ratio of deuterium is small, it can be easily determined whether target molecules are electrolytic hydrogen-containing molecules. Note that the threshold may be greater than 0 ppm.

As described above, the determination method according to the present embodiment determines whether target molecules including elemental hydrogen are electrolytic hydrogen-containing molecules including: hydrogen molecules produced by water electrolysis; or molecules produced using the hydrogen molecules as a raw material. The determination method determines that target molecules are electrolytic hydrogen-containing molecules when the abundance ratio of deuterium to light hydrogen in the target molecules is less than or equal to a predetermined threshold which is smaller than the abundance ratio of deuterium to light hydrogen in nature.

As described above, the abundance ratio of deuterium is smaller for: hydrogen molecules obtained by water electrolysis using a reaction rate difference; or molecules produced using the hydrogen molecules as a raw material. Thus, it can be determined that target molecules are electrolytic hydrogen-containing molecules when the abundance ratio of deuterium in the target molecules is smaller than that in nature. Thus, in the determination method according to the present embodiment, it is possible to confirm whether target molecules are hydrogen produced by water electrolysis, or molecules produced using the hydrogen as a raw material.

Water electrolysis can be performed using renewable energy, and it is possible to determine that target molecules are molecules produced using renewable energy in the determination method according to the present embodiment. That is, it becomes possible to construct traceability of molecules produced using renewable energy, by measuring the abundance ratio of deuterium to light hydrogen in target molecules. The method according to the present embodiment is particularly useful for sampling inspection at the time of receiving goods. Since the method according to the present embodiment is useful for sampling inspection, the quality can be assured by attaching an analysis result of produced electrolytic hydrogen-containing molecules, to a product as a quality record.

That is, the method according to the present embodiment may be a quality assurance method which assures that target molecules including elemental hydrogen are electrolytic hydrogen-containing molecules including hydrogen molecules produced by water electrolysis, or molecules produced using the hydrogen molecules as a raw material. The quality assurance method may assure that target molecules are electrolytic hydrogen-containing molecules when the abundance ratio of deuterium to light hydrogen in the target molecules is less than or equal to a predetermined threshold which is smaller than the abundance ratio of deuterium to light hydrogen in nature.

In the quality assurance method according to the present embodiment, the quality of target molecules can be confirmed by analyzing the abundance ratio of deuterium to light hydrogen in the target molecules when the target molecules are received. In the quality assurance method according to the present embodiment, the quality of target molecules to be shipped can be assured by analyzing the abundance ratio of deuterium to light hydrogen in the target molecules before shipping the target molecules. The quality of target molecules may be attached to products as an assurance certificate or a label

Note that the determination method may perform determination using a determination device including a determination unit. The determination device may include, for example, a measurement unit, a determination unit, and an output unit. The measurement unit may include a device for measuring the abundance ratio of deuterium to light hydrogen in target molecules. The measurement unit may include a mass spectrometer, for example. The measurement unit may be a combination of a mass spectrometer and a separation device, such as a gas chromatograph. The measurement unit may include a combination of a gas chromatograph and a detector.

The determination unit determines whether target molecules including elemental hydrogen are electrolytic hydrogen-containing molecules including: hydrogen molecules produced by water electrolysis; or molecules produced using the hydrogen molecules as a raw material. The determination unit determines that target molecules are electrolytic hydrogen-containing molecules when the abundance ratio of deuterium to light hydrogen in the target molecules is less than or equal to a predetermined threshold which is smaller than the abundance ratio of deuterium to light hydrogen in nature. The determination unit may determine that target molecules are electrolytic hydrogen-containing molecules when the abundance ratio of deuterium to light hydrogen in the target molecules obtained by the measurement unit is less than or equal to a predetermined threshold which is smaller than the abundance ratio of deuterium to light hydrogen in nature. An abundance ratio of deuterium data signal which has been output from the measurement unit is output to the determination unit, and the determination unit may acquire the data output from the measurement unit. The determination unit may be a CPU (central processing unit), or a computer including a memory, for example. The CPU reads a determination program stored in the memory, and can determine whether target molecules are electrolytic hydrogen-containing molecules, based on the abundance ratio of deuterium to light hydrogen in the target molecules, obtained by the measurement unit, and a threshold. The output unit outputs a determination result determined by the determination unit. Examples of the output unit include a monitor and a printer. For example, the output unit can output a determination result that target molecules are electrolytic hydrogen-containing molecules, or target molecules are not electrolytic hydrogen-containing molecules, to the output unit.

### (Electrolysis Device)

Next, an electrolysis device according to the present embodiment will be described. The electrolysis device according to the present embodiment can perform the water electrolysis described in the above embodiment. The electrolysis device according to the present embodiment may be a low temperature type water electrolysis device, or a medium-high temperature steam electrolysis device.

### (Low Temperature Type Water Electrolysis Device)

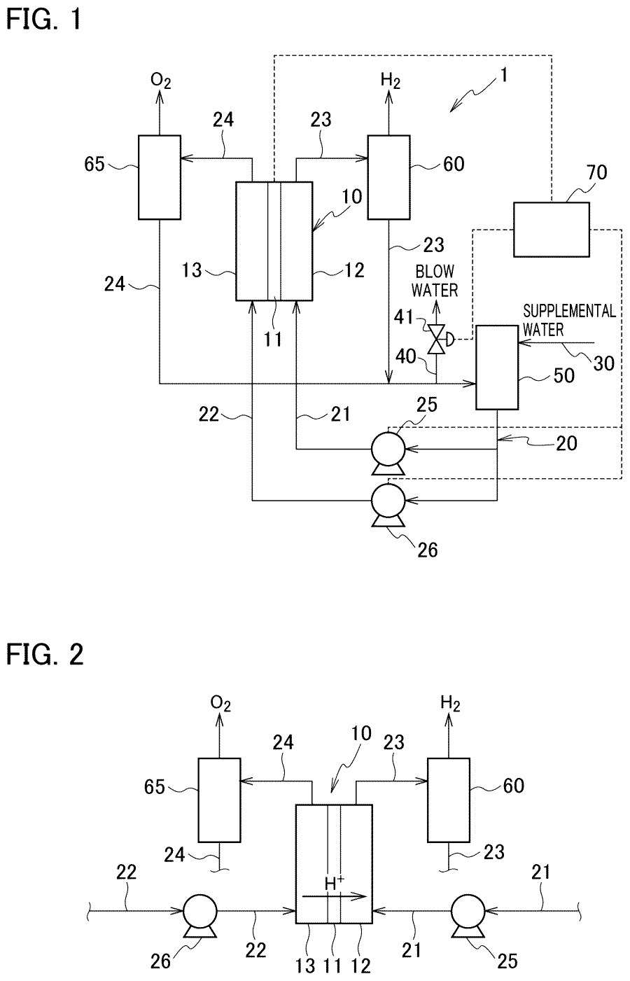

First, an example of a low temperature type water electrolysis device will be described with reference to FIG. 1. As illustrated in FIG. 1, an electrolysis device 1 according to the present embodiment includes an electrolytic cell 10, a circulation flow path 20, a water supply flow path 30, and a drainage flow path 40.

The electrolytic cell 10 electrolyzes water. Hydrogen and oxygen are produced by electrolysis of water. An electrolysis process in the electrolytic cell 10 may be alkaline type water electrolysis, solid polymer type water electrolysis, or a combination thereof. The electrolysis process in the electrolytic cell 10 may be PEM (proton exchange membrane) type water electrolysis, alkaline type water electrolysis, AEM (anion exchange membrane) type water electrolysis, or the like.

The electrolytic cell 10 includes a membrane 11, a cathode 12, and an anode 13. The electrolytic cell 10 includes a DC power supply (not illustrated) electrically connected to the cathode 12 and the anode 13, and water is electrolyzed by applying a voltage to the cathode 12 and the anode 13.

Water electrolyzed in the electrolytic cell 10 circulates in the circulation flow path 20. Since pure water is usually used as water supplied to the electrolysis device 1, pure water can be effectively utilized by circulating the water. The water supply flow path 30 and the drainage flow path 40 are connected to the circulation flow path 20. The water supply flow path 30 supplies pure water to the circulation flow path 20. The pure water may be water having an electrical resistivity of 0.1 Ω·cm or more at 25° C. The electrical resistivity of pure water may be 20 MΩ·cm or less, 10 M Ω·cm or less, or 1.5 MΩ·cm or less. The drainage flow path 40 drains part or all of the water in the circulation flow path 20 downstream of the electrolytic cell 10, and upstream of water supply via the water supply flow path 30. The drainage flow path 40 may be provided with a flow rate control device 41 for controlling the amount of water drained in the circulation flow path 20. The flow rate control device 41 can control the flow rate of water flowing in the drainage flow path 40, and the amount of water in the circulation flow path 20 drained from the drainage flow path 40 can be controlled by the flow rate control device 41. Thus, the abundance ratio of deuterium in hydrogen molecules produced in the electrolytic cell 10 can be adjusted. The flow rate control device 41 may be a flow rate control valve or the like.

The circulation flow path 20 may include a cathode-side water supply pipe 21, an anode-side water supply pipe 22, a cathode-side drain pipe 23, and an anode-side drain pipe 24. The electrolysis device 1 may include an electrolytic solution supply tank 50 provided in the circulation flow path 20, a hydrogen gas-liquid separator 60 provided in the circulation flow path 20, and an oxygen gas-liquid separator 65 provided in the circulation flow path 20. The cathode-side water supply pipe 21 is provided with a pump 25. The anode-side water supply pipe 22 is provided with a pump 26. The cathode-side drain pipe 23 is provided with the hydrogen gas-liquid separator 60. The anode-side drain pipe 24 is provided with the oxygen gas-liquid separator 65.

Supplemental water is supplied to the electrolytic solution supply tank 50 via the water supply flow path 30, and water to be electrolyzed in the electrolytic cell 10 is stored in the electrolytic solution supply tank 50. An outlet of the electrolytic solution supply tank 50 is connected to an inlet of the electrolytic cell 10 on a cathode 12 side via the cathode-side water supply pipe 21. Water is supplied from the electrolytic solution supply tank 50 to the cathode 12 side of the electrolytic cell 10 by operating the pump 25. The outlet of the electrolytic solution supply tank 50 is connected to an inlet of the electrolytic cell 10 on an anode 13 side via the anode-side water supply pipe 22. Water is supplied from the electrolytic solution supply tank 50 to the anode 13 side of the electrolytic cell 10 by operating the pump 26.

The outlet of the electrolytic cell 10 on the cathode 12 side is connected to the inlet of the electrolytic solution supply tank 50 via the cathode-side drain pipe 23. The cathode-side drain pipe 23 is provided with the hydrogen gas-liquid separator 60. The outlet of the electrolytic cell 10 on the anode 13 side is connected to the inlet of the electrolytic solution supply tank 50 via the anode-side drain pipe 24. The anode-side drain pipe 24 is provided with the oxygen gas-liquid separator 65. Water which has passed through the electrolytic cell 10 is supplied to the hydrogen gas-liquid separator 60 together with hydrogen gas produced at the cathode 12, and to the oxygen gas-liquid separator 65 together with oxygen gas produced at the anode 13.

In the hydrogen gas-liquid separator 60, hydrogen produced by electrolysis at the cathode 12, and water discharged without being electrolyzed in the electrolytic cell 10 are separated. The hydrogen separated by the hydrogen gas-liquid separator 60 is recovered and stored, for example, in a storage tank. In contrast, the water separated by the hydrogen gas-liquid separator 60 is supplied to the electrolytic solution supply tank 50 via the cathode-side drain pipe 23.

In the oxygen gas-liquid separator 65, oxygen produced by electrolysis at the anode 13, and water discharged without being electrolyzed in the electrolytic cell 10 are separated. The oxygen separated by the oxygen gas-liquid separator 65 is stored, for example, in a storage tank. In contrast, the water separated by the oxygen gas-liquid separator 65 is supplied to the electrolytic solution supply tank 50 via the anode-side drain pipe 24.

Water discharged from the electrolytic cell 10 without being electrolyzed in the electrolytic cell 10 is stored in the electrolytic solution supply tank 50, and the water circulates between the electrolytic cell 10 and the electrolytic solution supply tank 50.

The electrolysis device 1 may include a control unit 70. The control unit 70 may be electrically connected to at least one selected from the group consisting of the electrolytic cell 10, the pump 25, the pump 26, and the flow rate control device 41. The control unit 70 may control at least one of the applied voltage or the current density of the electrolytic cell 10. The control unit 70 may control the flow rate of water supplied to the electrolytic cell 10 by operating at least one of the pump 25 or the pump 26. The control unit 70 may control the flow rate of water in the circulation flow path 20 drained from the drainage flow path 40 by operating the flow rate control device 41. With these controls, the abundance ratio of deuterium to light hydrogen in hydrogen molecules produced in the electrolytic cell 10 can be adjusted.

Next, electrolytic reactions in a PEM type water electrolysis device, an alkaline type water electrolysis device, and an AEM type water electrolysis device will be described in detail.

### (PEM Type Water Electrolysis Device)

First, an example of a PEM type water electrolysis device will be described with reference to FIG. 2. As illustrated in FIG. 2, in the PEM type water electrolysis device, water is supplied to the anode 13 via the anode-side water supply pipe 22 of the electrolytic cell 10. At the anode 13, oxygen and hydrogen ions (H⁺) are produced from water by electrolysis. The membrane 11 is a PEM, and hydrogen ions (H⁺) pass through the membrane 11 to move from the anode 13 side to the cathode 12 side. At the cathode 12, hydrogen gas is produced from hydrogen ions that have passed through the membrane 11. Water may be supplied to the cathode 12 side via the cathode-side water supply pipe 21. Water need not be supplied via the cathode-side water supply pipe 21.

The rate at which deuterium ions (D⁺) pass through the membrane 11 is slower than the rate at which light hydrogen ions (H⁺) pass through the membrane 11. At the anode 13, the rate at which D⁺ is produced from HDO and D₂O is slower than the rate at which H⁺ is produced from H₂O. Thus, at the cathode 12, the amount of deuterium gas produced, such as HD gas and D₂ gas, is smaller than the amount of light hydrogen gas produced. Therefore, the abundance ratio of deuterium to light hydrogen in hydrogen molecules produced in the PEM type water electrolysis device is smaller than the abundance ratio of deuterium to light hydrogen in water supplied to the electrolytic cell 10.

### (Alkaline Type Water Electrolysis Device)

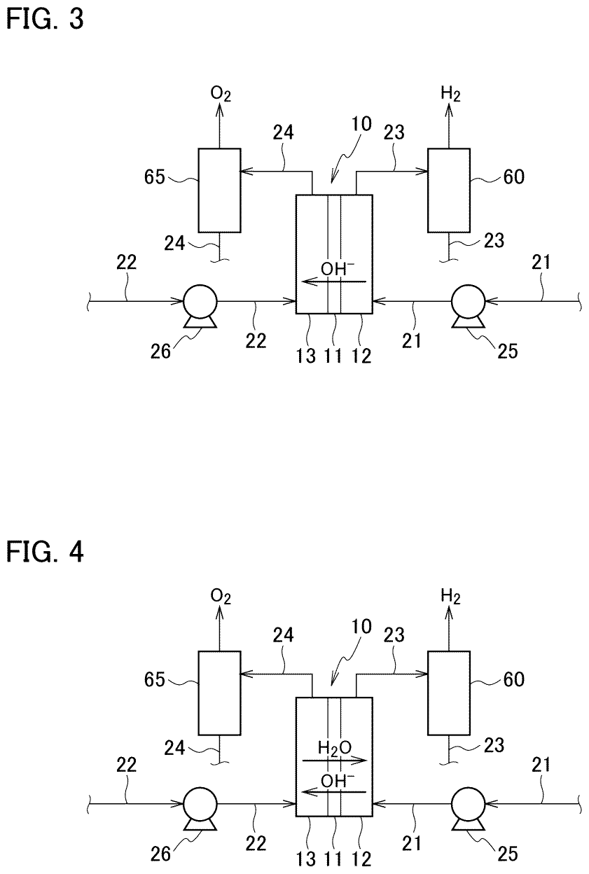

Next, an example of an alkaline type water electrolysis device will be described with reference to FIG. 3. As illustrated in FIG. 3, in the alkaline type water electrolysis device, water is supplied to the cathode 12 and the anode 13 of the electrolytic cell 10 via the cathode-side water supply pipe 21 and the anode-side water supply pipe 22, respectively. At the cathode 12, hydrogen and hydroxide ions (OH⁻) are produced from water by electrolysis. Hydroxide ions (OH⁻) pass through the membrane 11 to move from the cathode 12 side to the anode 13 side. At the anode 13, oxygen is produced from hydroxide ions (OH⁻) which have passed through the membrane 11. The membrane 11 is a diaphragm which may include at least one selected from the group consisting of polysulfone, PTFE (polytetrafluoroethylene), asbestos, polyolefin, and an anion exchange membrane (AEM). The anion exchange membrane may be a resin having a quaternary ammonium group and an imidazolium group. Alkaline water passing through the electrolytic cell 10 may include an aqueous solution of an alkali metal hydroxide. The alkali metal hydroxide may include at least one of sodium hydroxide or potassium hydroxide.

The rate at which OD⁻, HD gas, and D₂ gas are produced from HDO and D₂O at the cathode 12 is slower than the rate at which OH⁻ and H₂ gas are produced from H₂O. Thus, at the cathode 12, the amount of deuterium gas produced, such as HD gas and D₂ gas, is smaller than the amount of light hydrogen gas generated. Therefore, the abundance ratio of deuterium to light hydrogen in hydrogen molecules produced in the alkali type water electrolysis device is smaller than the abundance ratio of deuterium to light hydrogen in water supplied to the electrolytic cell 10.

### (AEM Type Water Electrolysis Device)

Next, an example of an AEM type water electrolysis device will be described with reference to FIG. 4. As illustrated in FIG. 4, in the AEM water electrolysis device, water is supplied to the anode 13 of the electrolytic cell 10 via the anode-side water supply pipe 22. Water passes through the membrane 11, which is an AEM, to move from the anode 13 side to the cathode 12 side. At the cathode 12, hydrogen and hydroxide ions (OH⁻) are produced by electrolysis from water that has passed through the membrane 11. Hydroxide ions (OH⁻) produced at the cathode 12 pass through the membrane 11 to move from the cathode 12 side to the anode 13 side. At the anode 13, oxygen and water are produced from hydroxide ions (OH⁻) that have passed through the membrane 11. Water may be supplied to the cathode 12 side through the cathode-side water supply pipe 21. Water need not be supplied through the cathode-side water supply pipe 21.

The rate at which HDO and D₂O pass through the membrane 11 is slower than the rate at which H₂O passes through the membrane 11. At the cathode 12, the rate at which HD gas and D₂ gas are produced from HDO and D₂O is slower than the rate at which H₂ gas is produced from H₂O. Thus, at the cathode 12, the amount of deuterium gas produced, such as HD gas and D₂ gas, is smaller than the amount of light hydrogen gas produced. Therefore, the abundance ratio of deuterium to light hydrogen in hydrogen molecules produced in the AEM type water electrolysis device is smaller than the abundance ratio of deuterium to light hydrogen in water supplied to the electrolytic cell 10.

### (Medium-High Temperature Steam Electrolysis Device)

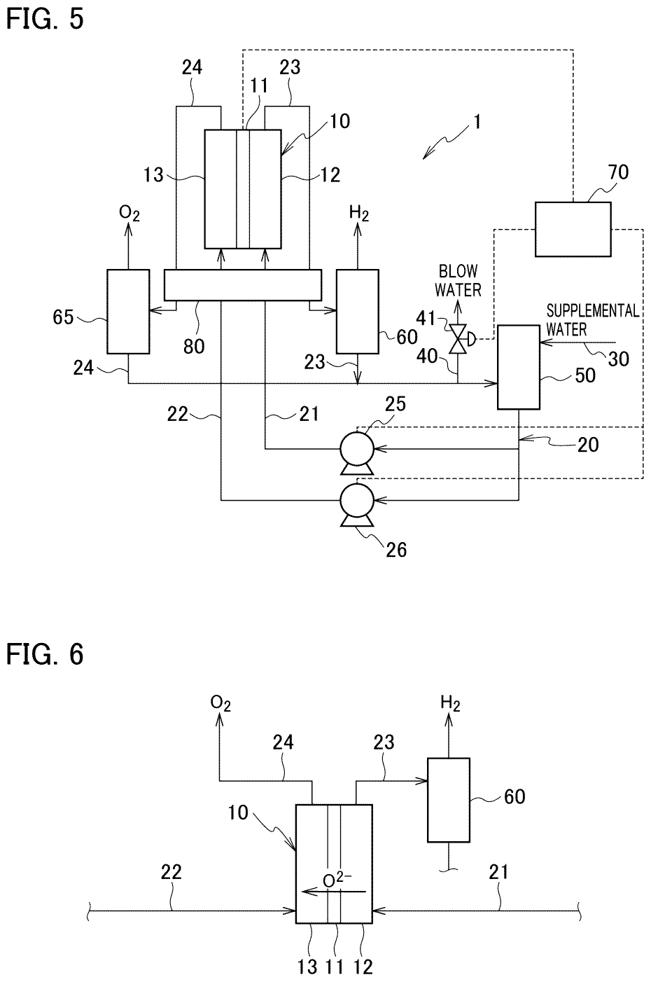

Next, an example of a medium-high temperature steam electrolysis device will be described with reference to FIG. 5. In the electrolysis device 1 according to the present embodiment, an electrolysis process in the electrolytic cell 10 may be SOEC (solid oxide electrolysis cell) type water electrolysis, PCEC (protonic ceramic electrolysis cell) type water electrolysis, or a combination thereof. As illustrated in FIG. 5, the electrolysis device 1 according to the present embodiment further includes a heat exchanger 80 provided in the circulation flow path 20. Other than this, the electrolysis device is the same as the low temperature type water electrolysis device illustrated in FIG. 1, and therefore the description thereof will be omitted.

The heat exchanger 80 exchanges heat between water supplied to the electrolytic cell 10 and water drained from the electrolytic cell 10. The heat exchanger 80 may include a first heat exchanger provided to extend over the cathode-side water supply pipe 21 and the cathode-side drain pipe 23, and a second heat exchanger provided to extend over the anode-side water supply pipe 22 and the anode-side drain pipe 24. The first heat exchanger can exchange heat between water supplied to the cathode 12 side of the electrolytic cell 10 with water discharged from the cathode 12 side of the electrolytic cell 10. The second heat exchanger can exchange heat between water supplied to the anode 13 side of the electrolytic cell 10 with water discharged from the anode 13 side of the electrolytic cell 10. Note that in place of the heat exchanger 80, a heater (not illustrated) for heating water supplied to the electrolytic cell 10 may be provided in at least one of the cathode-side water supply pipe 21 or the anode-side water supply pipe 22.

Next, electrolytic reactions in the SOEC type water electrolysis device and the PCEC type water electrolysis device will be described in detail.

### (SOEC Type Water Electrolysis Device)

An example of an SOEC type water electrolysis device will be described with reference to FIG. 6. As illustrated in FIG. 6, in the SOEC type water electrolysis device, steam is supplied to the cathode 12 of the electrolytic cell 10 via the cathode-side water supply pipe 21. At the cathode 12, hydrogen gas and oxygen ions (O²⁻) are produced from water vapor by electrolysis. Oxygen ions (O²⁻) pass through the membrane 11 to move from the cathode 12 side to the anode 13 side. At the anode 13, oxygen gas is produced from oxygen ions (O²⁻) that have passed through the membrane 11.

At the cathode 12, the rate at which HD gas and D₂ gas are produced from HDO and D₂O is slower than the rate at which H₂ gas is produced from H₂O. Thus, at the cathode 12, the amount of deuterium gas produced, such as HD gas and D₂ gas, is smaller than the amount of light hydrogen gas produced. Therefore, the abundance ratio of deuterium to light hydrogen in hydrogen molecules produced in the SOEC type water electrolysis device is smaller than the abundance ratio of deuterium to light hydrogen in water supplied to the electrolytic cell 10.

### (PCEC Type Water Electrolysis Device)

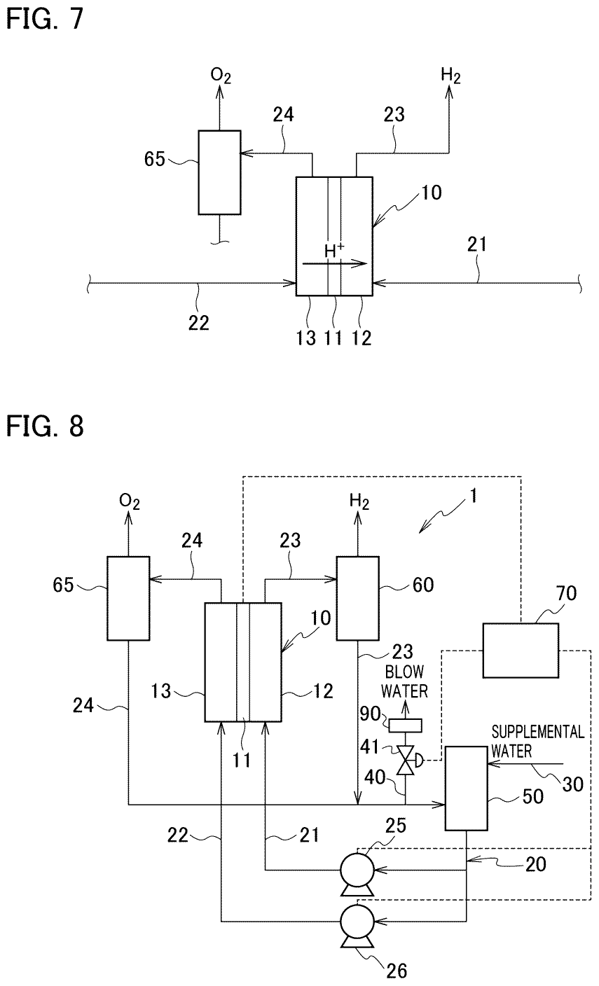

Next, an example of a PCEC type water electrolysis device will be described with reference to FIG. 7. As illustrated in FIG. 7, in the PCEC type water electrolysis device, steam is supplied to the anode 13 via the anode-side water supply pipe 22. At the anode 13, oxygen gas and hydrogen ions (H⁺) are produced from steam by electrolysis. Hydrogen ions (H⁺) pass through the membrane 11 to move from the anode 13 side to the cathode 12 side. At the cathode 12, hydrogen gas is produced from hydrogen ions (H⁺) that have passed through the membrane 11.

The rate at which deuterium ions (D⁺) pass through the membrane 11 is slower than the rate at which light hydrogen ions (H⁺) pass through the membrane 11. At the anode 13, the rate at which D⁺ is produced from HDO and D₂O is slower than the rate at which H⁺ is produced from H₂O. Thus, at the cathode 12, the amount of deuterium gas produced, such as HD gas and D₂ gas, is smaller than the amount of light hydrogen gas produced. Therefore, the abundance ratio of deuterium to light hydrogen in hydrogen molecules produced in the PCEC type water electrolysis device is smaller than the abundance ratio of deuterium to light hydrogen in water supplied to the electrolytic cell 10.

As described above, in any of the electrolysis processes, the abundance ratio of deuterium to light hydrogen in hydrogen molecules produced is smaller than the abundance ratio of deuterium to light hydrogen in water supplied to the electrolytic cell 10. In contrast, the abundance ratio of deuterium in water discharged from the electrolytic cell 10 is larger than the abundance ratio of deuterium in water supplied to the electrolytic cell 10. In the electrolysis device 1 according to the present embodiment, part or all of the water in the circulation flow path 20 is drained through the drainage flow path 40, and pure water is supplied to the circulation flow path 20 through the water supply flow path 30. Thus, the abundance ratio of deuterium in water flowing in the circulation flow path 20 becomes smaller by dilution, and the abundance ratio of deuterium in hydrogen molecules produced in the electrolytic cell 10 becomes even smaller. Therefore, the abundance ratio of deuterium to light hydrogen in hydrogen molecules produced by water electrolysis in the electrolytic cell 10 is smaller than the abundance ratio of deuterium to light hydrogen in nature.

Note that the water supplied to the electrolytic cell 10 may be alkaline water, and the electrolysis device 1 may further include a membrane separator 90, as illustrated in FIG. 8. The membrane separator 90 may be provided in the drainage flow path 40. The membrane separator 90 may include a permeable membrane that selectively passes water in alkaline water therethrough. A semipermeable membrane selectively passes water in alkaline water therethrough. The semipermeable membrane passes water in alkaline water therethrough, but does not pass metal ions therethrough, such as sodium ions and potassium ions. Thus, only water can be discharged without discharging alkaline water to the outside of the circulation flow path 20.

The semipermeable membrane may include at least one selected from the group consisting of a flat membrane, a hollow fiber membrane, and a spiral membrane. The pore size of the semipermeable membrane may be such that water molecules pass through, but sodium ions in water to be treated do not pass through. The pore size of the semipermeable membrane may be 0.5 nm or more, or 1 nm or more. The pore size of the semipermeable membrane may be 10 nm or less, 5 nm or less, or 2 nm or less. The semipermeable membrane may be a reverse osmosis membrane (RO membrane). The semipermeable membrane may include at least one selected from the group consisting of cellulose acetate, polyacrylonitrile, polysulfone, polyethersulfone, polyethylene, polypropylene, polyvinylidene fluoride, and ceramic.

Next, the abundance ratio of deuterium to light hydrogen was evaluated by simulation under various operating conditions of the electrolysis device illustrated in FIG. 1. The operating conditions are listed in Table 1, and the abundance ratio of deuterium to light hydrogen is listed in Table 2.

TABLE 1

| Operation conditions | | 1 | 2 | 3 | 4 | 5 | 6 | 7 | 8 | 9 | 10 | 11 | 12 |
|---|---|---|---|---|---|---|---|---|---|---|---|---|---|
| Water utilization ratio | — | 0.1 | | | | | | | | 0.5 | | | |
| Separation factor | — | 600 | | | | 10 | | | | 600 | | | |
| Blow ratio | — | 0.05 | 0.1 | 0.2 | 0.5 | 0.05 | 0.1 | 0.2 | 0.5 | 0.05 | 0.1 | 0.2 | 0.5 |

| | | |
|---|---|---|
| Amount of hydrogen | Nm³/h | 8000 |
| gas generated | t/d | 17.1 |

TABLE 2

| Operation conditions | | 1 | 2 | 3 | 4 | 5 | 6 | 7 | 8 | 9 | 10 | 11 | 12 |
|---|---|---|---|---|---|---|---|---|---|---|---|---|---|
| Supplemental water | mol-ppm | 150 | | | | | | | | | | | |
| Electrolytic cell | mol-ppm | 430 | 284 | 210 | 165 | 365 | 261 | 202 | 164 | 1542 | 825 | 448 | 225 |
| supply water | | | | | | | | | | | | | |
| Hydrogen gas | mol-ppm | 0.7 | 0.5 | 0.3 | 0.3 | 36.5 | 26.1 | 20.2 | 16.4 | 2.57 | 1.38 | 0.75 | 0.38 |

In Table 1, a water utilization ratio is a volume ratio of the amount of electrolysis consumption water to the amount of electrolytic cell supply water. The electrolytic cell supply water is water that is supplied to the electrolytic cell 10 through the cathode-side water supply pipe 21 and the anode-side water supply pipe 22. The electrolysis consumption water is water to be consumed by electrolysis in the electrolytic cell 10.

A separation factor is a value expressed by the following formula:

$$α = ( {[ D ]}_{L} ) / ( {[ D ]}_{G} )$$

In the above formula, a represents the separation factor, [D]_{L} represents the abundance ratio of deuterium to light hydrogen in electrolytic cell supply water, and [D]_{G} represents the abundance ratio of deuterium to light hydrogen in hydrogen gas (hydrogen molecules) produced in the electrolytic cell 10.

The separation factor can be controlled using an applied voltage and a current density as operating variables, which are operating conditions of the electrolytic cell 10. The smaller the applied voltage, the smaller the abundance ratio of deuterium to light hydrogen in hydrogen molecules produced. The smaller the current density, the smaller the abundance ratio of deuterium to light hydrogen in hydrogen molecules produced. Furthermore, the separation factor can be controlled by selecting the metal specie of electrocatalyst. The separation factor tends to decrease in the order Cu>Fe>Ni>Ag>Au>Pt>Sn.

A blow ratio is the volume ratio of a flow rate of blow water to a flow rate of produced water. The produced water is water discharged from the electrolytic cell 10 without being electrolyzed in the electrolytic cell 10. The blow water is water discharged from the drainage flow path 40. The flow rate is the amount of water per unit time. The amount of hydrogen gas generated is the amount of hydrogen gas (hydrogen molecules) generated by electrolysis in the electrolytic cell 10.

In Table 2, supplemental water is pure water supplied to the circulation flow path 20 via the water supply flow path 30. The abundance ratio of deuterium to light hydrogen in pure water is set to 150 ppm. Electrolytic cell supply water is water supplied to the electrolytic cell 10 as described above. The electrolytic cell supply water is a mixture of supplemental water and water that circulates in the circulation flow path 20 without produced water being discharged as blow water. In this example, the abundance ratio of deuterium to light hydrogen in the electrolytic cell supply water is larger than the abundance ratio of deuterium to light hydrogen in the supplemental water.

As listed in Table 2, the abundance ratio of deuterium to light hydrogen in hydrogen gas produced in the electrolytic cell 10 can be controlled depending on operating conditions of the electrolysis device. Specifically, the lower the water utilization ratio is, the more the abundance ratio of deuterium can be reduced. The larger the separation factor is, the more the abundance ratio of deuterium can be reduced. The larger the blow ratio is, the more the abundance ratio of deuterium can be reduced.

Thus, at least one selected from the group consisting of a water utilization ratio, a separation factor, and a blow ratio may be controlled. The water utilization ratio is the ratio of the amount of water consumed by electrolysis in the electrolytic cell 10 to the amount of water supplied to the electrolytic cell. The separation factor is the ratio of the abundance ratio of deuterium to light hydrogen in water supplied to the electrolytic cell 10 to the abundance ratio of deuterium to light hydrogen in hydrogen molecules produced in the electrolytic cell 10. The blow ratio is the ratio of the flow rate of water discharged in the drainage step to the flow rate of water drained from the electrolytic cell 10. By producing hydrogen molecules having a low abundance ratio of deuterium by the above-described operation, hydrogen molecules derived from fossil fuels can be more easily distinguished. These controls may be performed by the control unit 70 by controlling the electrolytic cell 10, the pump 25, the pump 26, and the flow rate control device 41.

When the abundance ratio of deuterium in target molecules is measured, and the abundance ratio of deuterium in hydrogen molecules is within a range calculated based on operation conditions illustrated in Table 1, it can be easily determined that the target molecules are electrolytic hydrogen-containing molecules. In addition, the quality of the target molecules can be assured by checking the abundance ratio of deuterium specifications issued by manufacturers of fuels derived from renewable energy or raw materials.

As described above, the electrolysis device 1 according to the present embodiment includes the electrolytic cell 10 for electrolyzing water, the circulation flow path 20 for circulating water to be electrolyzed in the electrolytic cell 10, and the water supply flow path 30 for supplying pure water to the circulation flow path 20. The electrolysis device 1 includes the drainage flow path 40 for draining part or all of the water in the circulation flow path 20 downstream of the electrolytic cell 10 and upstream of the water supply via the water supply flow path 30. The abundance ratio of deuterium to light hydrogen in hydrogen molecules produced by water electrolysis in the electrolytic cell 10 is smaller than the abundance ratio of deuterium to light hydrogen in nature.

The electrolysis method according to the present embodiment includes an electrolysis step for electrolyzing water in the electrolytic cell 10, and a water supply step for supplying pure water to the circulation flow path 20, which circulates water to be electrolyzed in the electrolytic cell 10. The electrolysis method includes a drainage step for draining part or all of the water in the circulation flow path 20 downstream of the electrolytic cell 10 and upstream of water supply in the water supply step. The abundance ratio of deuterium to light hydrogen in hydrogen molecules produced by water electrolysis in the electrolytic cell 10 is smaller than the abundance ratio of deuterium to light hydrogen in nature.

In the electrolysis device and the electrolysis method according to the present embodiment, water is electrolyzed in the electrolytic cell 10. The abundance ratio of deuterium to light hydrogen in hydrogen molecules produced in the electrolytic cell 10 is smaller than the abundance ratio of deuterium to light hydrogen in water supplied to the electrolytic cell 10. In contrast, the abundance ratio of deuterium in water discharged from the electrolytic cell 10 is larger than that of water supplied to the electrolytic cell 10. In the electrolysis device 1 according to the present embodiment, part or all of the water in the circulation flow path 20 is drained via the drainage flow path 40, and pure water is supplied to the circulation flow path 20 via the water supply flow path 30. Thus, the abundance ratio of deuterium in water flowing in the circulation flow path 20 is reduced by dilution, and the abundance ratio of deuterium in hydrogen molecules produced in the electrolytic cell 10 is also reduced. Therefore, the abundance ratio of deuterium to light hydrogen in hydrogen molecules produced by water electrolysis in the electrolytic cell 10 is smaller than the abundance ratio of deuterium to light hydrogen in nature.

In contrast, when water in the circulation flow path 20 is not drained, all of the water supplied to the electrolytic cell 10 is finally electrolyzed, and thus the abundance ratio of deuterium to light hydrogen in hydrogen molecules produced in the electrolytic cell 10 is the same as the abundance ratio of deuterium to light hydrogen in nature. Thus, the electrolysis device and the electrolysis method according to the present embodiment can reduce the abundance ratio of deuterium to light hydrogen in hydrogen molecules compared with the case where water in the circulation flow path 20 is not drained. Moreover, the abundance ratio of deuterium to light hydrogen in molecules produced can be reduced by producing molecules, such as ammonia and hydrocarbons, by using hydrogen molecules whose abundance ratio of deuterium is reduced, as a raw material.

Therefore, in the electrolysis device and the electrolysis method according to the present embodiment, it can be easily confirmed whether target molecules are hydrogen produced by water electrolysis, or molecules produced using the hydrogen as a raw material.

The entire contents of Japanese Patent Application No. 2022-175572 (filed Nov. 1, 2022) are incorporated herein by reference.

Although several embodiments have been described, modifications and variations are possible based on the above-described disclosure. All components of the above embodiments and all features described in the claims may be individually extracted and combined as long as they do not conflict with each other.

The present disclosure can contribute, for example, to goal 7 “Ensure access to affordable, reliable, sustainable and modern energy for all”, goal 12 “Ensure sustainable consumption and production patterns”, and goal 13 “Take urgent action to combat climate change and its impacts” of the Sustainable Development Goals (SDGs) led by the United Nations.

What is claimed is:

1. A determination method for determining whether or not target molecules including elemental hydrogen are electrolytic hydrogen-containing molecules which include:
hydrogen molecules produced by water electrolysis; or molecules produced using the hydrogen molecules as a raw material, the method comprising:
determining that the target molecules are the electrolytic hydrogen-containing molecules when an abundance ratio of deuterium to light hydrogen in the target molecules is less than or equal to a predetermined threshold which is smaller than an abundance ratio of deuterium to light hydrogen in nature.

2. The determination method according to claim 1, wherein the target molecules are hydrogen molecules, ammonia, or a hydrocarbon.

3. An electrolysis device comprising:
an electrolytic cell that electrolyzes water;
a circulation flow path where water to be electrolyzed in the electrolytic cell circulates;
a water supply flow path that supplies pure water to the circulation flow path; and
a drainage flow path that drains part or all of the water in the circulation flow path, downstream of the electrolytic cell and upstream of the water supply via the water supply flow path, wherein
an abundance ratio of deuterium to light hydrogen in hydrogen molecules produced by water electrolysis in the electrolytic cell is smaller than an abundance ratio of deuterium to light hydrogen in nature.

4. The electrolysis device according to claim 3, wherein a flow rate control device that controls an amount of water drained in the circulation flow path is provided in the drainage flow path.

5. The electrolysis device according to claim 3, wherein
water supplied to the electrolytic cell is alkaline water, and
the electrolysis device further includes a membrane separator that is provided in the drainage flow path and includes a permeable membrane which selectively passes water in the alkaline water therethrough.

6. An electrolysis method according to claim 3, comprising:
an electrolysis step of electrolyzing water in the electrolytic cell;
a water supply step of supplying pure water to the circulation flow path that circulates water to be electrolyzed in the electrolytic cell; and
a drainage step of draining part or all of the water in the circulation flow path, downstream of the electrolytic cell and upstream of the water supply in the water supply step.

7. The electrolysis method according to claim 6, wherein at least one selected from the group consisting of: a ratio of an amount of water consumed by electrolysis in the electrolytic cell to an amount of water supplied to the electrolytic cell; a ratio of an abundance ratio of deuterium to light hydrogen in water supplied to the electrolytic cell to an abundance ratio of deuterium to light hydrogen in hydrogen molecules produced in the electrolytic cell; and a ratio of a flow rate of water drained in the drainage step to a flow rate of water discharged from the electrolytic cell, is controlled.

8. A determination device comprising:
a determination unit that determines whether or not target molecules including elemental hydrogen are electrolytic hydrogen-containing molecules which include hydrogen molecules produced by water electrolysis, or molecules produced using the hydrogen molecules as a raw material, wherein
the determination unit determines that the target molecules are the electrolytic hydrogen-containing molecules when an abundance ratio of deuterium to light hydrogen in the target molecules is less than or equal to a predetermined threshold which is smaller than an abundance ratio of deuterium to light hydrogen in nature.

9. The determination device according to claim 8, further comprising:
a measurement unit that measures the abundance ratio of deuterium to light hydrogen in the target molecules;
a determination unit that determines that the target molecules are the electrolytic hydrogen-containing molecules when the abundance ratio of deuterium to light hydrogen in the target molecules obtained by the measurement unit is less than or equal to a predetermined threshold which is less than the abundance ratio of deuterium to light hydrogen in nature; and
an output unit that outputs a determination result determined by the determination unit.
